(19) United States
(12) Patent Application Publication
(10) Pub. No.: US 2012/0016476 A1
(43) Pub. Date: Jan. 19, 2012

(54) INTERVERTEBRAL IMPLANT WITH A HINGE END CAP

(75) Inventors: Bret M. Wilfong, Hernando, MS (US); Michael J. Merves, Memphis, TN (US)

(73) Assignee: Warsaw Orthopedic, Inc., Warsaw, IN (US)

(21) Appl. No.: 12/836,704

(22) Filed: Jul. 15, 2010

(51) Int. Cl. A61F 2/44

(52) U.S. Cl. 623/17.11

(57) ABSTRACT

Devices and methods for spacing apart vertebral members. The implant may include a body and an end cap. Connectors may extend outward from the body to attach with the end cap. The end cap may include a hinged design to securely attach to the body. The hinged design may include first and second sections that are connected at a hinge. The implant may also include a second end cap positioned at the opposite end of the body. The second end cap may be the same or different than the first end cap.

### BACKGROUND

The present application is directed to devices and methods for stabilizing vertebral members, and more particularly, to intervertebral implants and methods of use for replacing an intervertebral disc, vertebral member, or combination of both to distract and/or stabilize the spine.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various conditions may lead to damage of the intervertebral discs and/or the vertebral members. The damage may result from a variety of causes including but not limited to a specific event such as trauma, a degenerative condition, a tumor, or infection. Damage to the intervertebral discs and vertebral members can lead to pain, neurological deficit, and/or loss of motion.

Various procedures include replacing the entirety or a section of a vertebral member, the entirety or a section of an intervertebral disc, or both. One or more replacement implants may be inserted to replace the damaged vertebral members and/or discs. The implants are configured to be inserted into the intervertebral space and contact against the remaining adjacent vertebral members. The implants reduce or eliminate the pain and neurological deficit, and increase the range of motion.

The curvature of the spine and general shapes of the vertebral members may make it difficult for the implants to adequately contact the adjacent vertebral members. There is a need for implants configurable to match the spinal anatomy and space for secure contact when implanted into an intervertebral space.

### SUMMARY

The present application is directed to implants for insertion into an intervertebral space between first and second vertebral members. The implants may include a body with a first end and a second end, first and second extensions that may each extend axially outward from the first end, and a pivoting end cap that may be attached to the first end of the body. The end cap may have a first section with a first aperture that receives the first extension, a second section with a second aperture that receives the second extension, and a hinge that pivotally connects the first and second sections together. The hinge may be configured to position the second section at a variety of angular positions relative to the first section. A first securing feature at the first aperture may attach the first section to the body. A different second securing feature at the second aperture may attach the second section to the body.

The implant may also include a body with a first end and a second end, and first and second extensions that may each extend axially outward from the first end. Each of the extensions may include a stem that extends outward from the body and an enlarged head that is spaced away from the body by the stem. A pivoting end cap may be attached to the first end of the body. The end cap may have a first section with a first aperture that receives the first extension, a second section with a second aperture that receives the second extension, and a hinge that pivotally connects the first and second sections together. The first aperture may have an elongated shape with a wide section that is wider than the head of the first extension and a narrow section adjacent to the wide section that is narrower than the head of the extension and wider than the stem of the first extension. The body may have a flexible portion that defines a portion of the second aperture. The flexible portion may be movable between a first position with the second aperture having a first width that is greater than the head of the second extension and a second position with the second aperture having a second width that is smaller than the head of the second extension.

A method of assembling an implant that is inserted into an intervertebral space between first and second vertebral members may include positioning an end cap at an end of a body with the end cap having first and second sections that are hinged together and movable between a first pivoted orientation and a second substantially flat orientation. While the end cap is in the first orientation, the method may include inserting a first extension that extends outward from the body into a corresponding first aperture in the first section of the end cap. The method may include rotating the end cap relative to the body and moving the first extension from a wide section in the first aperture to a narrow section in the first aperture and securing the first section to the body. While the first plurality of extensions are inserted in the plurality of first apertures, the method may include pivoting the second section of the end cap relative to the first section and into contact with the body. The method may include inserting a second aperture in the second section over a second extension that extends outward from the body. The method may include securing the second extension in the second aperture and securing the second section to the body.

The various aspects of the various embodiments may be used alone or in any combination, as is desired.

### BRIEF DESCRIPTION OF THE DRAWINGS

FIG. 1 is a schematic diagram of an implant according to one embodiment positioned in an intervertebral space between vertebral members.

FIG. 2 is a perspective view of an implant with an end cap in an open orientation according to one embodiment.

FIG. 3 is a perspective view of an implant with an end cap in a closed orientation according to one embodiment.

FIG. 4 is an exploded perspective view of an end cap and a portion of a body according to one embodiment.

FIG. 5 is a perspective view of a base of a body according to one embodiment.

FIG. 6 is a side view of an end cap in an open orientation on a base of a body according to one embodiment.

FIG. 7 is a perspective view of a base of a body with an end cap in an open orientation according to one embodiment.

FIG. 8 is a top view of an end cap attached to a body according to one embodiment.

### DETAILED DESCRIPTION

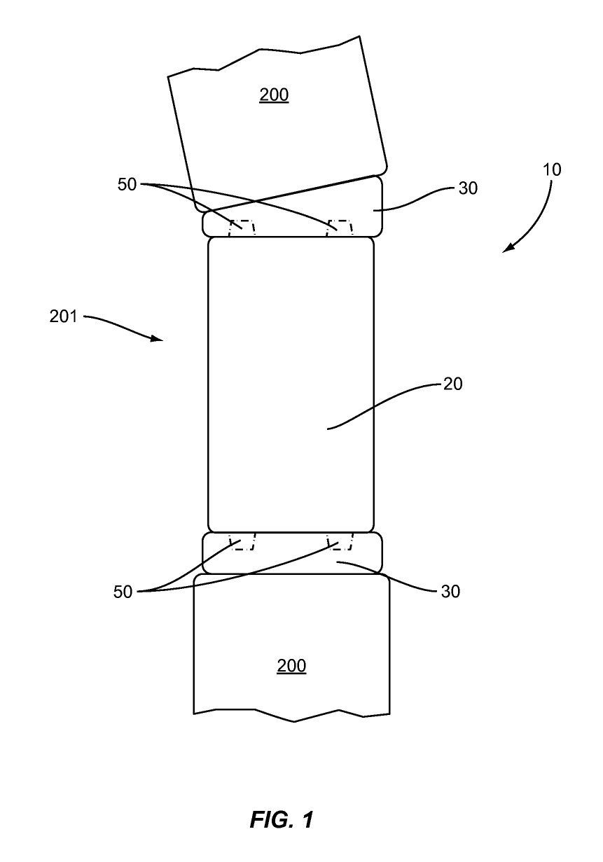

The present application is directed to intervertebral implants for spacing apart vertebral members. FIG. 1 illustrates an implant 10 positioned within an intervertebral space 201 formed between vertebral members 200. The implant 10 includes a body 20 and an end cap 30. Connectors 50 extend outward from the body 20 to attach with the end cap 30. The end cap 30 is sized and shaped to accommodate the anatomy of the vertebral members 200 and the intervertebral space 201. The end cap 30 includes a hinged design to securely attach to the body 20. The implant 10 may also include a second end cap 30 positioned at the opposite end of the body 20. The second end cap 30 may be the same or different than the first end cap 30.

The attachable end cap 30 allows the implant 10 to be configured to accommodate a variety of different applications. Various different end caps 30 may be attached to the body 20 with the various end caps 30 having different angular shapes, sizes, materials, etc. The hinged design prevents the end cap 30 from rotating relative to the body 20 which could cause the end cap 30 to become detached.

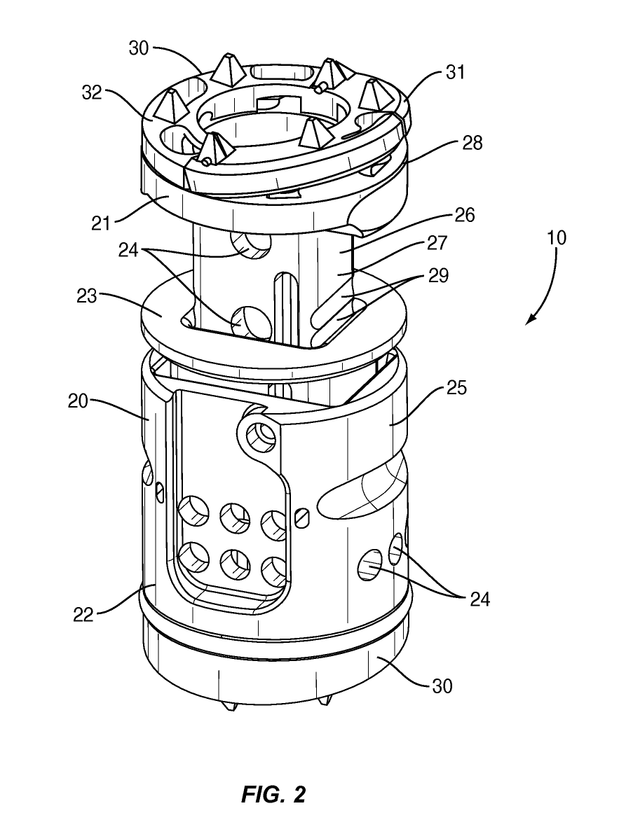

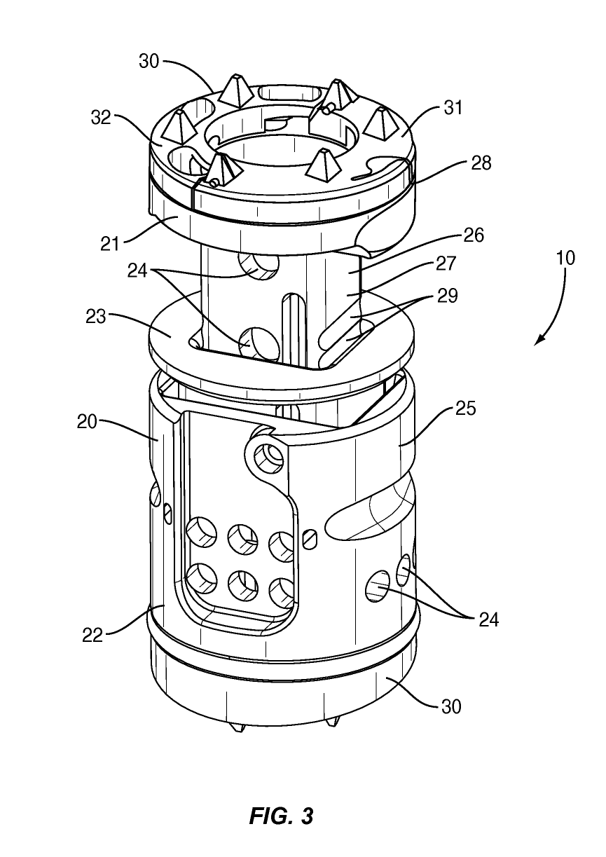

FIGS. 2 and 3 illustrate an implant 10 with a body 20 and an end cap 30. The body 20 in combination with the end cap 30 is sized to fit within the intervertebral space 201. FIG. 2 includes the end cap 30 in an open orientation with a first section 31 pivoted away from the body 20. The end cap 30 is in the open orientation prior to insertion into the intervertebral space 201. FIG. 3 includes the end cap 30 in a closed orientation with the first section 31 pivoted towards the body 20. The end cap 30 is moved to the closed orientation prior to insertion into the intervertebral space 201.

The body 20 includes an elongated shape with a first end 21 and a second end 22. The body 20 may also include a hollow interior sized to receive bone growth material. One or more apertures 24 may extend through the body 20 to the hollow interior.

The body 20 may be constructed from a single section with a fixed height measured between the first and second ends 21, 22. The body 20 may also be constructed of two or more relatively movable sections to adjust the height. FIGS. 2 and 3 include the body 20 with a first outer section 25 and a second inner section 26. The outer section 25 includes a hollow interior and the inner section 26 includes a neck 27 and a base 28. The neck 27 is sized to fit within and axially move along the hollow interior to adjust a height of the body 20. The neck 27 further includes a plurality of scallops 29 that extend along the length. Both the outer and inner sections 25, 26 may be hollow and include one or more apertures to receive bone growth material.

A securing mechanism 23 may secure the sections 25, 26 together to fix the height. In one embodiment, the securing mechanism 23 is configured to receive one or more cylindrical rods (not illustrated) that seat within the plurality of scallops 29 that extend along the neck 27. U.S. Patent Publication No. 2008/0114467 discloses embodiments of an implant that may be used with end caps and include a multiple-section body and a locking mechanism and is herein incorporate by reference in its entirety.

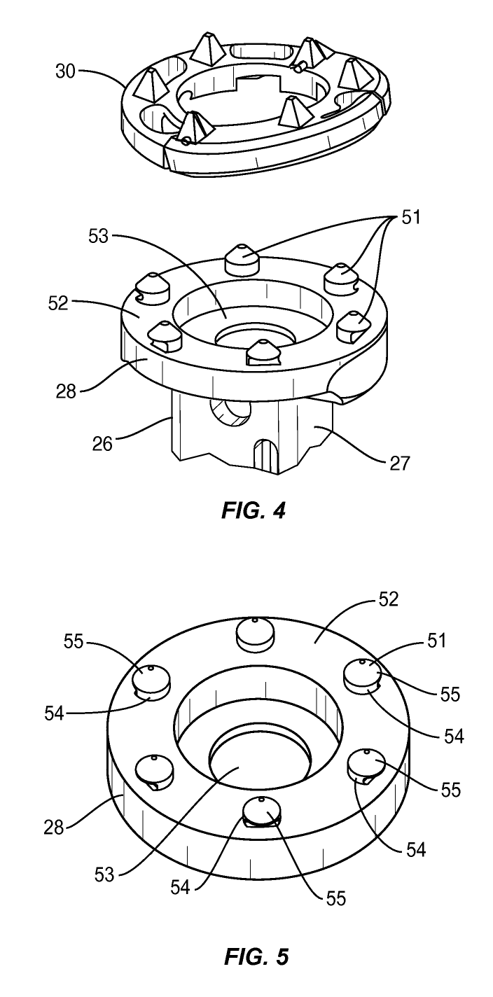

The body 20 is configured to receive the end cap 30. FIGS. 4, 5, and 6 include the base 28 that forms the first end 21 and has a support surface 52 that extends around a periphery of a central aperture 53. In this embodiment, the support surface 52 is substantially flat, although other embodiments may include a variety of different surface configurations. A plurality of extensions 51 extend axially outward from the support surface 52. The extensions 51 are evenly spaced around the periphery and are sized to extend into apertures in the end cap 30 as will be explained below. FIG. 5 includes an embodiment with six extensions 51 that are spaced about 60° apart around the periphery of the base 28. Other embodiments may include two or more extensions 51.

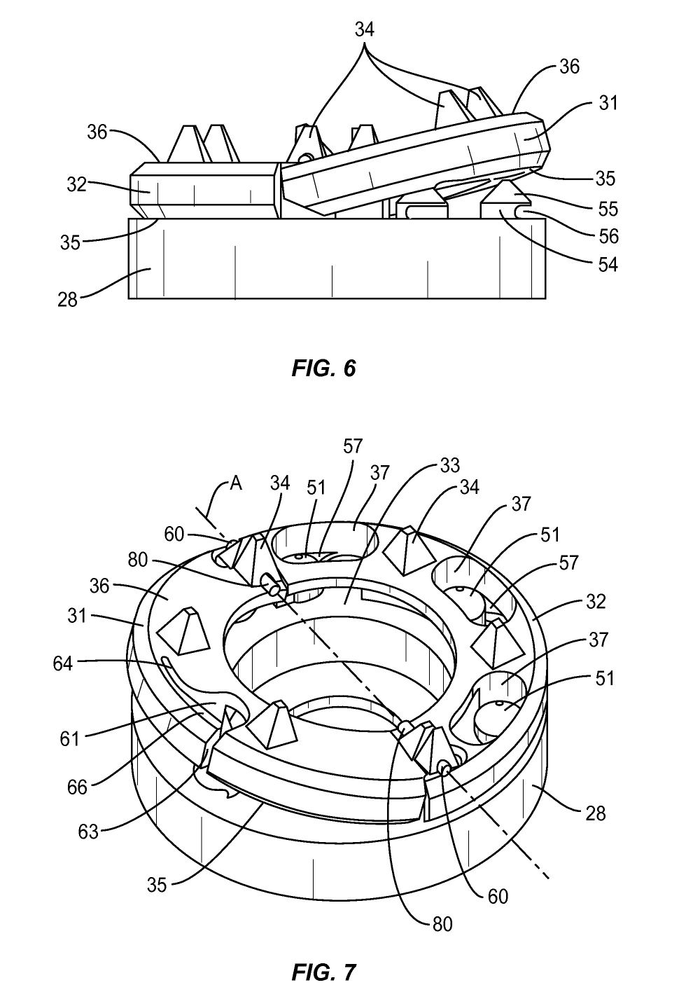

The extensions 51 include a stem 54 that extends axially outward from the surface 52 and are capped with a head 55. The head 55 includes a tapered shape that terminates at a tip. The tip facilitates attachment of the end cap 30. The tip may also be shaped to directly contact against the vertebral member 200 when the implant 10 is used without an end cap 30, or when the extension 51 extends beyond the end cap 30. As best illustrated in FIGS. 5 and 6, the stem 54 includes a smaller width than the head 55 forming an undercut section 56. The depth of the undercut section 56 may be the same or different for each of the extensions 51. The undercut sections 56 may face radially outward from the aperture 53. One or more extensions 51 may include a stem 54 that has substantially the same width as the head 55. In one embodiment, the width of the stem 54 is equal to the widest part of the head 55.

The end cap 30 is attached to the body 20. As illustrated in FIGS. 6, 7, and 8, the end cap 30 includes an inner side 35 that faces towards the body 20, and an opposing outer side 36. The height of the end cap 30 measured between the sides 35, 36 may vary across the width to accommodate the size of the intervertebral space 201 and the shape of the adjacent vertebral member 200. In one embodiment as illustrated in FIG. 1, the end cap 30 has a wedge shape with a first outer side having a smaller height than an opposing second outer side. This shape may accommodate the lordotic or kyphotic shape of the spine depending upon the vertebral level at which the implant 10 is positioned in the patient.

In one embodiment, the inner sides 35 of each of the first and second sections 31, 32 are substantially flat to abut against the surface 52 of the base 28. The outer side 36 may also be flat, or may include various other configurations to facilitate contact with the vertebral member 200. In one embodiment with the end cap 30 in the open orientation, the inner side 35 of the first section 35 is substantially perpendicular to the inner side 35 of the second section 36. In one embodiment with the end cap 30 in the closed orientation, the inner sides 35 of the first and second sections 35, 36 are aligned in a co-planar arrangement. One or more teeth 34 may extend outward from the outer side 36 to bite into the vertebral member 200.

The end cap 30 may include a variety of outer geometric shapes when viewed from the end. FIG. 8 includes the end cap 30 having a circular shape. Other shapes include but are not limited to polygonal and crescent-shaped. The end cap 30 may also include a central aperture 33 that may also have various geometric shapes.

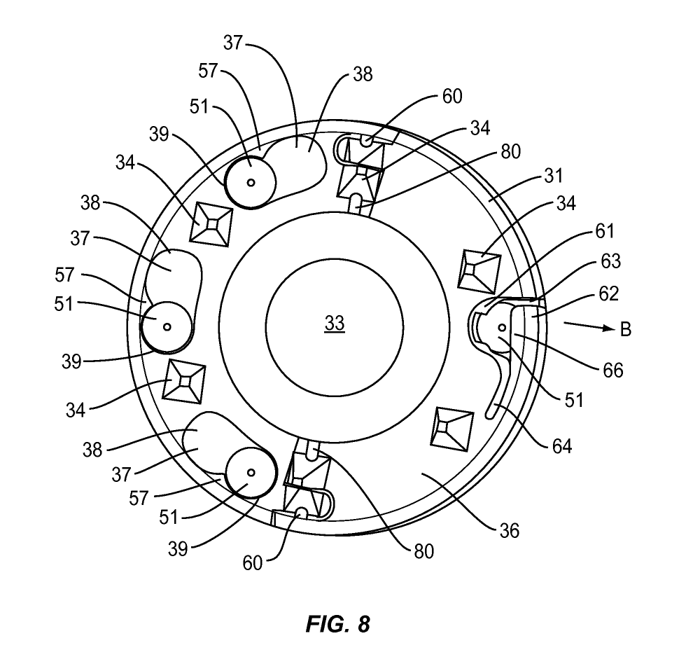

The first and second sections 31, 32 may be substantially similar. The embodiments illustrated in FIGS. 6, 7, and 8, include each of the sections 31, 32 having an arcuate shape each in the shape of a C. The shapes are configured to align to form an overall circular shape for the end cap 30. In one embodiment as illustrated in FIGS. 7 and 8, each section 31, 32 includes a tongue that fits into a corresponding receptacle in the opposing section. In some embodiments, the overall width of the end cap 30 is equal to a width of the base 28. Other embodiments include the end cap 30 having a greater width or a smaller width than the base 28. The central aperture 33 of the end cap 30 may align with the hollow interior of the body 20.

The first and second sections 31, 32 are connected together by a hinge 80 that forms a pivot axis A. The hinge 80 provides for the first section 31 to pivot about the axis A relative to the second section 32. The hinge 80 may include a single pin that extends through both sections 31, 32, or two or more pins 60 as illustrated in FIGS. 7 and 8. In embodiments with a single pin, the pin may extend over a majority or entirety of the end cap 30. The sections 31, 32 may also be configured for the pins 60 to extend through one or more of the teeth 34. As illustrated in FIGS. 7 and 8, the first section 31 includes a first portion of one or more teeth 34, and the second section 32 includes a remainder portion of the teeth 34. These teeth 34 are completed when the sections 31, 32 are connected together with the pins 60 and the sections 31, 32 are in the closed orientation.

The first section 31 also includes one or more apertures 37 that receive corresponding extensions 51 on the body 20. The apertures 37 are spaced around the first section 31 to accommodate the positioning of the extensions 51. In one embodiment, the apertures 37 are contained with the first section 31 and include a continuous sidewall. The apertures 37 include a wide section 38 and a narrow section 39. The wide section 38 includes a greater width measured between opposing surfaces than the narrow section 39. The wide section 38 is wider than the head 55 of the extension 51 to allow the end cap 30 to be mounted onto the body 20. The narrow section 38 is narrower than the head 55 for the end cap 30 to be secured to the body 20.

A lip 57 extends into the aperture 37 from the sidewall to form the edge of the narrow section 39. The lip 57 is configured to fit within the undercut section 56 of the extension 51 to attach the end cap 30 to the body 20. The lip 57 is recessed below the outer surface 36 of the end cap 30. This positioning locates the lip 57 to fit under the head 55 of the extension 51 and within the undercut section 56 when the first section 31 is secured to the body 20. The number of apertures 37 in the first section 31 may vary from a single aperture 37 to multiple apertures 37. FIGS. 7 and 8 include embodiments with three apertures 37.

The second section 32 includes at least one aperture 61 that receives an extension 51. These aperture 61 acts in combination with the one or more apertures 37 in the first section 31 to maintain attachment of the end cap 30. The aperture 61 is sized to receive the extension 51. A pair of slots 63, 64 are in communication with the aperture and form a flexible finger 62 in the second section 32. The first slot 63 extends between the aperture 61 and the exterior edge of the second section 32. The second slot 64 extends from the aperture 61 and along the exterior edge. The material of the second section 32, and the position and shapes of the aperture 61 and slots 63, 64 cause the finger 62 to be flexible. This flexibility causes the finger 62 to move outward to enlarge the size of the aperture 61 to receive the head 55. Specifically, as the second section 32 pivots downward towards the body 20 along the pivot axis A, the finger 62 contacts against and slides along the tapered head 55 of the extension 51. This movement causes the finger 62 to flex outward in the direction of arrow B illustrated in FIG. 8. Once the finger 62 moves beyond the head 55, the finger 62 flexes inward in an opposite direction and contacts against the extension 51.

In one embodiment, a lip 66 extends from the finger 62 inward into the aperture 61. The lip 66 is positioned below the outer surface 36 and includes a height to fit within the undercut section 56. The inner surface of the lip 66 slides along the tapered head 55 as the second section 32 is pivoted onto the body 20. Once the lip 66 moves beyond the head 55, the finger 62 flexes inward and the lip 66 moves under the head 55 and within the undercut section 56 to attach the second section 32.

The second section 32 may include one or more apertures 61. FIGS. 7 and 8 illustrate an embodiment with a single aperture 61. Other embodiments may include two or more apertures 61. In multiple aperture 61 embodiments, the apertures 61 may include the same or different shapes and/or sizes.

Assembling the implant 10 includes initially determining the type of end cap 30 that is to be attached to the body 20. The end cap 30 may be selected based on the size of the intervertebral space 201 and the anatomy of the vertebral members 200.

The proper end cap 30 is determined and positioned at the first end 21 of the body 20. The one or more apertures 37 in the first section 31 of the end cap 30 are aligned with the one or more corresponding extensions 51 that axially extend outward from the surface 52 of the body 20. The end cap 30 is moved towards the body 20 with the extensions 51 inserting into the wide sections 38 of the apertures 37. In one embodiment, the first section 31 of the end cap 30 is moved towards the body 20 until the inner surface 35 contacts against the surface 52 of the body 20. Another embodiment does not include the inner surface 35 contacting against the surface 52.

During attachment of the first section 31, the second section 32 of the end cap 30 is pivoted upward relative to the first section 31. This prevents the second section 32 from contacting against the body 20 and possibly interfering with the movement of the first section 31.

Once the first section 31 is mounted onto the body 20 with the one or more extensions 51 in the corresponding one or more apertures 37, the end cap 30 is rotated relative to the body 20. In the embodiment illustrated in FIG. 8, the end cap 30 is rotated in a counter-clockwise direction. This rotation moves the extensions 51 into the narrow sections 39 of the apertures 37. This movement causes the lips 57 that extend into the apertures 37 to be moved underneath the heads 55 and into the undercut sections 56. The end cap 30 may be rotated until the head 55 contacts against the end of the apertures 37 or just until the lips 57 move underneath the heads 55.

In one embodiment, the extensions 51 on the body 20 and the apertures 37 in the end caps 30 each have the same size and shape. Therefore, rotation of the end cap 30 causes each aperture 37 to be secured around the corresponding extension 51 in a similar manner. In other embodiments, one or more of the extensions 51 and/or apertures 37 include a different shape and/or size. This causes differing amounts of contact between the various extensions 51 and apertures 37.

FIGS. 7 and 8 include the first section 31 including multiple apertures 37. Other embodiments may include the first section 31 including a single aperture 37, two apertures 37, or four or more apertures 37.

Once the first section 31 is secured to the body 20, the second section 32 is pivoted downward along the axis A towards the surface 52 of the body 20. The position of the extensions 51 are arranged such that the aperture 61 in the second section 32 aligns with the corresponding extension 51 when the first section 31 is secured to the body 20. As the second section 32 is pivoted downward, the extension 51 moves into the corresponding aperture 61. Continued movement causes the head 55 of the extension 51 to contact against the finger 62 adjacent to the aperture 61. The tapered shape of the head 55 causes the finger 62 to flex outward away from the central aperture 33 of the end cap 30. This outward movement increases the width of the aperture 61.

The second section 32 continues to move down towards the surface 52 of the body 20 until moving beyond the head 55. Once beyond the head 55, the finger 62 flexes inward towards the central aperture 33. This movement causes the lip 66 on the inner side of the finger 62 to move under the head 55 and into the undercut section 56. This positioning secures the extension 51 within the aperture 61 and secures the second section 32 to the body 20.

The finger 62 at the aperture 61 assumes a first position for a width of the aperture 61 to be smaller than the head 55 of the extension 51. This first position occurs when no external forces are being applied to the finger 62. The force of the second section 32 moving downward towards the body 20 causes the outward flexing of the finger 62. In one embodiment, the first position includes the aperture 61 having a greater width than the stem 54 of the extension 51. In this embodiment, the finger 62 is spaced away from the stem 54 when it seats under the head 55 of the extension 51. In another embodiment, the aperture 61 includes a smaller width than the stem 54 and the finger 62 contacts against the stem 54 to further secure the end cap 30 to the body 20.

FIGS. 7 and 8 include the second section 32 including a single aperture 61 and the body 20 including a single extension 51. Other embodiments may include multiple apertures 61 that receive multiple extensions 51.

FIGS. 7 and 8 include the end cap 30 having the same number of apertures 37, 61 as corresponding extensions 51. Specifically, FIG. 8 includes three apertures 37 and three extensions 51 in the first section 31, and one aperture 61 and one extension 51 in the second section 32. Other embodiments may include the end cap 30 including more apertures 37, 61 than extensions 51. These embodiments include one or more empty apertures 37, 61.

The different securement mechanisms on the first and second sections 31, 32 maintain the end cap 30 secured to the body 20. The configuration of the one or more apertures 61 in the second section 32 and the corresponding extensions 51 reduces or prevents rotational movement of the end cap 30 relative to the body 20 that may cause the first section 31 to be detached from the body 20. Likewise, the configuration of the one or more apertures 37 and the corresponding extensions 51 in the first section 32 prevents or reduces movement of the end cap 30 relative to the body 20 that may cause detachment of the second section 32.

The second section 32 may also include a variety of other securing mechanisms. In one embodiment, the finger 62 is a deformable member that may be deformed by the surgeon. After the extension 51 is within the corresponding aperture 61, the finger 62 is deformed to secure the end cap 30. Other mechanisms for securing the end cap 30 to the body 20 may include a adhesives such as epoxy, a taper lock, surface configurations between the body 20 and end cap 30, such as roughened surfaces, dissimilar materials, and shape differences.

The body 20 and end cap 30 may be constructed from a variety of different materials. Examples include but are not limited to stainless steels, titanium, PEEK, plastics, and various other biocompatible materials. The body 20 and end cap 30 may be constructed from the same or different materials. The pins 60 may also be constructed from one or more of these materials.

The implants 10 may be implanted within a living patient for the treatment of various spinal disorders. The implant 10 may also be implanted in a non-living situation, such as within a cadaver, model, and the like. The non-living situation may be for one or more of testing, training, and demonstration purposes.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. An implant for insertion into an intervertebral space between first and second vertebral members, the implant comprising:
a body with a first end and a second end;
first and second extensions that each extend axially outward from the first end;
a pivoting end cap attached to the first end of the body, the end cap having a first section with a first aperture that receives the first extension, a second section with a second aperture that receives the second extension, and a hinge that pivotally connects the first and second sections together, the hinge configured to position the second section at a variety of angular positions relative to the first section;
a first securing feature at the first aperture to attach the first section to the body; and
a second securing feature at the second aperture to attach the second section to the body, the second securing feature being different than the first securing feature.

2. The implant of claim 1, wherein the first and second extensions include an enlarged head and an undercut section between the head and the first end of the body, the undercut section including a smaller width than the head and being configured to accommodate the first and second securing features respectively.

3. The implant of claim 2, wherein the heads of the first and second extensions include a tapered shape with a narrow tip that faces away from the body.

4. The implant of claim 2, wherein the undercut sections of the first and second extensions each face radially outward away from a center of the body.

5. The implant of claim 1, wherein the first securing feature includes a lip that extends outward from a sidewall of the first aperture and forms a narrow section in the first aperture, the lip being spaced away from an outer surface of the end cap.

6. The implant of claim 1, wherein the second securing feature includes a flexible finger that forms a sidewall of the second aperture, the finger being radially flexible to adjust a width of the second aperture.

7. The implant of claim 1, wherein the end cap has a circular shape with a central aperture that aligns with an aperture in the body when the end cap is mounted to the body.

8. An implant for insertion into an intervertebral space between first and second vertebral members, the implant comprising:
a body with a first end and a second end;
first and second extensions that each extend axially outward from the first end, each of the extensions including a stem that extends outward from the body and an enlarged head that is spaced away from the body by the stem;
a pivoting end cap attached to the first end of the body, the end cap with a first section with a first aperture that receives the first extension, a second section with a second aperture that receives the second extension, and a hinge that pivotally connects the first and second sections together;
the first aperture having an elongated shape with a wide section that is wider than the head of the first extension and a narrow section adjacent to the wide section that is narrower than the head of the extension and wider than the stem of the first extension;
the body having a flexible portion that defines a portion of the second aperture, the flexible portion being movable between a first position with the second aperture having a first width that is greater than the head of the second extension and a second position with the second aperture having a second width that is smaller than the head of the second extension.

9. The implant of claim 8, wherein the end cap includes a circular shape with a central aperture and the hinge includes a first pin that extends through a first set of openings in each of the first and second sections and is positioned on a first side of the aperture and a second pin that extends through a second set of openings in each of the first and second sections and is positioned on an opposing second side of the aperture.

10. The implant of claim 8, wherein the first section includes a third aperture and the body includes a third extension that fits within the third aperture when the end cap is attached to the body, the third aperture having the same shape as the first aperture and the third extension having the same shape as the first extension.

11. The implant of claim 8, wherein the end cap includes a wedge shape with a first outer side that is taller than an opposing second outer side.

12. The implant of claim 8, wherein the heads of the first and second extensions include a tapered shape with a tip that faces away from the body.

13. The implant of claim 8, wherein the flexible portion includes a lip recessed below an outer surface of the end cap, the lip extends into the second aperture.

14. The implant of claim 8, wherein the flexible portion assumes the second position prior to be attached to the body.

15. The implant of claim 8, further comprising a second end cap attached to the second end of the body.

16. A method of assembling an implant that is inserted into an intervertebral space between first and second vertebral members, the method comprising:
positioning an end cap at an end of a body, the end cap having first and second sections that are hinged together and movable between a first pivoted orientation and a second substantially flat orientation;
while the end cap is in the first orientation, inserting a first extension that extends outward from the body into a corresponding first aperture in the first section of the end cap;
rotating the end cap relative to the body and moving the first extension from a wide section in the first aperture to a narrow section in the first aperture and securing the first section to the body;
while the first plurality of extensions are inserted in the plurality of first apertures, pivoting the second section of the end cap relative to the first section and into contact with the body;
inserting a second aperture in the second section over a second extension that extends outward from the body; and
securing the second extension in the second aperture and securing the second section to the body.

17. The method of claim 16, further comprising pivoting the second section of the end cap relative to the first section and aligning inner sides of the first and second sections of the end cap in a planar arrangement.

18. The method of claim 16, further comprising inserting a third extension that extends outward form the body into a corresponding third aperture in the first section of the end cap.

19. The method of claim 16, further comprising inserting the second extension into the second aperture and deforming the second aperture.

20. The method of claim 19, further comprising positioning a lip that extends from the sidewall of the second aperture under an enlarged head of the second extension.
